SEQUENCE LISTING (20 sequences)

SEQ ID NO: 1 - DNA, 18 bp, Artificial Sequence
  Primer
gccatccacgctgttttg

SEQ ID NO: 2 - DNA, 43 bp, Artificial Sequence
  Primer
ctgaccatggtgtccggatggataagagccaaaggggtgtgcc

SEQ ID NO: 3 - DNA, 40 bp, Artificial Sequence
  Primer
ccatccggacaccatggtcagcaagggcgaggagctgttc

SEQ ID NO: 4 - DNA, 32 bp, Artificial Sequence
  Primer
ctgacgtgcatgcgtcacttgtacagctcgtc

SEQ ID NO: 5 - DNA, 28 bp, Artificial Sequence
  Primer
gatatcgaattccaccatgagcgagctg

SEQ ID NO: 6 - DNA, 29 bp, Artificial Sequence
  Primer
ctaatggtcgacgattatctgtgccccag

SEQ ID NO: 7 - DNA, 20 bp, Artificial Sequence
  Primer
cgcgccaccagacataatag

SEQ ID NO: 8 - DNA, 36 bp, Artificial Sequence
  Primer
cagaattcatgcatcaccatgagcgagctgatcaag

SEQ ID NO: 9 - DNA, 36 bp, Artificial Sequence
  Primer
atgatatcgcatgcgattatctgtgccccagtttgc

SEQ ID NO: 10 - DNA, 30 bp, Artificial Sequence
  Primer
catcatgaattcgccaccatggtgagcaag

SEQ ID NO: 11 - DNA, 35 bp, Artificial Sequence
  Primer
ctgatatcacgcgttacttgtacagctcgtccatg

SEQ ID NO: 12 - DNA, 21 bp, Artificial Sequence
  Primer
ggcttttttgttagacgctag

SEQ ID NO: 13 - DNA, 47 bp, Artificial Sequence
  Primer
ctccctctggtcctcctgatgcagcagccccggggagcatgtcaagg

SEQ ID NO: 14 - DNA, 44 bp, Artificial Sequence
  Primer
gcatcaggaggaccagagggaggttccatggccaagttgaccag

SEQ ID NO: 15 - DNA, 37 bp, Artificial Sequence
  Primer
cataatcagctgcggccgcgaaatctcgtagcacgtg

SEQ ID NO: 16 - DNA, 30 bp, Artificial Sequence
  Primer
ctaataaccggtctggagacgccatccacg

SEQ ID NO: 17 - DNA, 21 bp, Artificial Sequence
  Primer
gtgaacagctcctcgcccttg

SEQ ID NO: 18 - DNA, 33 bp, Artificial Sequence
  Primer
catacagaattcgacaccatggggaaggtgaag

SEQ ID NO: 19 - DNA, 77 bp, Artificial Sequence
  Primer
ctgatatcacgcgtcacgtagaatcgagtccgagtagagggttaggtataggcttgccctccttggaggccatgtgg

SEQ ID NO: 20 - DNA, 26 bp, Artificial Sequence
  Primer
catgatgaattcgacggtgactgcag

(19) United States
(12) Patent Application Publication
(10) Pub. No.: US 2016/0040186 A1
(43) Pub. Date: Feb. 11, 2016

(54) DNA CONSTRUCT AND METHOD FOR TRANSGENE EXPRESSION

(71) Applicant: Xiaoyun Liu, Allen, TX (US)

(72) Inventor: Xiaoyun Liu, Allen, TX (US)

(21) Appl. No.: 14/454,697

(22) Filed: Aug. 7, 2014

(51) Int. Cl. C12N 15/86; C12N 15/80; C12N 15/81

(52) U.S. Cl. CPC C12N 15/86; C12N 15/815; C12N 15/80

(57) ABSTRACT

This invention relates to a DNA construct that is capable of expressing a desired transgene in a trackable manner. The construct comprises in 5′ to 3′ downstream direction: a promoter; a fluorescent reporter gene positioned within an intron defined by a 5′-donor splice site comprising a splice donor sequence and a 3′-acceptor splice site comprising a splice acceptor sequence; a desired gene and a transcription terminator. A method of producing a transgenic host cell having a desired product is also disclosed.

### CROSS-REFERENCE TO RELATED APPLICATIONS

### U.S. Patent Documents

| | | |
|---|---|---|
| 5,654,168 | August 1997 | Bujard et al.. |
| 5,891,718 | August 1999 | Hobart et al.. |
| U.S. Pat. No. 5,561,053 A | October 1996 | Crowley. |
| 2009/0305343 A1 | December 2009 | Fallot et al.. |

### OTHER PUBLICATIONS

Pelletier et al., Internal initiation of translation of eukaryotic mRNA directed by a sequence derived from poliovirus RNA. Nature. 1988; 334:320-325.
Mizuguchi et al., IRES-dependent second gene expression is significantly lower than cap-dependent first gene expression in a bicistronic vector. Mol. Ther. 2000; 1: 376-382.
Mansha et al., Problems encountered in bicistronic IRES-GFP expression vectors employed in functional analyses of GC-induced genes. Mol. Biol. Rep. 2012; 39: 10227-10234.
Szymczak et al., Correction of multi-gene deficiency in vivo using a single ‘self-cleaving’ 2A peptide-based retroviral vector. Nat. Biotechnol. 2004; 22: 589-594.
Lengler et al., FMDV-2A sequence and protein arrangement contribute to functionality of CYP2B1-reporter fusion protein. Anal. Biochem. 2005; 343: 116-124.
Hasegawa et al., Efficient multicistronic expression of a transgene in human embryonic stem cells. Stem Cells. 2007; 25: 1707-1712.
Mount, A catalogue of splice junction sequences. Nucleic Acids Res. 1982; 10: 459-472.
Senapathy et al., Splice junctions, branch point sites, and exons: sequence statistics, identification, and applications to genome project. Methods Enzymol. 1990; 183: 252-278.
Winey et al., A synthetic intron in a naturally intronless yeast pre-tRNA is spliced efficiently in vivo. Mol. Cell Biol. 1989; 9: 329-331.
Gatermann et al., Introduction of functional artificial introns into the naturally intronless ura4 gene of Schizosaccharomyces pombe. Mol. Cell Biol. 1989; 9: 1526-1535.
Fallot, et al., Alternative-splicing-based bicistronic vectors for ratio-controlled protein expression and application to recombinant antibody production. Nucleic Acids Res. 2009; 37: e134.
Sambrook et al., 1989, Molecular Cloning: A Laboratory Manual, Cold Spring Harbor Press: Cold Spring Harbor N.Y; selected unnumbered pages.

### BACKGROUND OF THE INVENTION

1. Field of the Invention

This invention relates to a DNA construct capable of expressing a desired transgene in a trackable manner, a method of producing transgenic host cells having a desired product.

2. Description of Background and Related Art

Transgene expression involves several steps. In the first step, a recombinant DNA segment having the desired gene insert is constructed. Such a DNA segment, also known as “expression cassette” consists of in order from 5′ to 3′, a promoter, the desired gene, and a transcription terminator. In the second step, the expression cassette is inserted into the host cell population by various means known in the art. In order to facilitate identification of transduced cells, a second expression cassette having a promoter, a gene encoding a selectable marker, and a transcription terminator is also transduced into the host cells together with the desired construct. In the third step, cells are grown in a given culture medium having a selective reagent. Host cells surviving the selective pressure are isolated and amplified. In the last step, transgenic host cells are analyzed for the expression of the desired gene by various means known in the art.

Despite general success of such gene transfer and selection methods, production of transgenic host cells having the desired product tends to be cost-ineffective. The known methods for inserting a DNA segment into eukaryotic host cells are highly inefficient. Of the cells in a given culture, only a small proportion takes up exogenous DNA and even a smaller percentage of the cells express the transgene. In addition, a large proportion of host cells surviving selective pressure do not have the desired product, because the desired gene is independent of the transcription regulatory elements of the selectable marker. Hence, a number of single cell clones surviving selective pressure need to isolate and amplify independently to large population prior to determining the desired product. However, expansion of cells and identification of the desired product using the methods known in the art are time-consuming and expensive.

In order to improve efficiency for transgene expression, certain genes are chosen as reporters because their expression are easily identified and measured. In most applications, reporter genes are used as indicators of whether a certain gene has been taken up by or expressed in the cell or organism population. In this regard, genes coding fluorescent proteins have been widely incorporated into expression vectors as reporters. Such a method is very convenient because the presence of a fluorescent reporter is easily visualized by using a fluorescent microscope that requires limited number of cells.

One attempt is to use a gene chimera comprising a fluorescent reporter and the desired gene. In such a way, a host cell having a phenotype of fluorescent reporter also expresses the desired gene. This method has been widely used in the study of cellular localization of the protein of interest. However, the product chimera may have improper function.

Another method utilizes an internal ribosome entry site (IRES) to link the desired gene to a fluorescent reporter, while a single promoter and transcription terminator regulate the transcription of both genes. Upon entry into a host cell, the genes are transcribed together as a single RNA molecule (Pelletier et al., Nature. 334: 320-325, 1988). In the subsequent process, the single RNA transcript translates to unlinked desired product and fluorescent protein in a Cap- or an IRES-mediated manner. A major drawback of such a DNA construct is that Cap- and IRES-mediated translation is adversely affected by each other (Mizuguchi et al. Mol. Ther. 1: 376-382, 2000; Mansha et al., Mol. Biol. Rep. 39: 10227-10234, 2012). Hence, the correlation of the fluorescent reporter and desired product tends unpredictable. The other method is to insert a host cell with a DNA chimera comprising a fluorescent reporter and a desired gene, while a self-cleavable peptide (Szymczak et al., Nat. Biotechnol. 22: 589-594, 2004) is incorporated to link the genes. Using a mechanism of ribosome skipping, such a DNA construct is capable of translating to unlinked fluorescent reporter and the desired product. However, this DNA construct may produce a protein chimera that has improper function because of the unpredictable efficiency for ribosome skipping (Lengler et al., Anal. Biochem. 343: 116-124, 2005; Hasegawa et al., Stem Cells. 25: 1707-1712. 2007). In addition, It has also been observed that using such a DNA construct, host cell clones with a phenotype of the selectable marker may not express the desired gene.

A single gene in eukaryotic cells is capable of producing multiple protein products through a process of alternative splicing. In this process, particular exons or introns of a gene may be included within, or excluded from, the final, processed messenger RNA (mRNA) produced from that gene. Hence, a single gene is capable of producing multiple mRNA transcripts. Consequently, each mRNA transcript may produce a protein product that contains differences in their amino acid sequence.

The mechanism of alternative intron splicing is useful for integrating multiple genes in a single expression cassette. In the U.S. Pat. No. 5,561,053, dihydrofolate reductase, or dhfr gene, a selectable marker, was positioned within a spliceable intron followed by a desired gene. The single pre-mRNA molecule produced by such a vector comprised both DHFR and the desired gene transcripts. Subsequently, the intron was either spliced out for expression of the desired product or maintained in mRNA to express the intronic DHFR, resulting in a selectable phenotype characteristic. This method is useful for selecting host cells expressing a desired product at high levels. However, it may be limited to DHFR-deficient cells. In addition, it is not suitable for selecting host cells having transgene expression in an inducible manner, because the intronic gene is a selectable marker.

The US patent 2009/0305343 A1 describes a method to express two proteins using the mechanism of alternative splicing. The gene coding one protein of interest is inserted in a spliceable intron, while the other gene locates at downstream of the 3′-terminus of the splice acceptor sequence. This vector is capable of expressing two proteins with fine-tuned ratios in the transgenic cell population. However, the patent does not describe their correlation in single host cell clones. Therefore, the capability of such a vector to track the presence of a desired product remains unknown.

### SUMMARY

With the above drawbacks in mind, one aim of the present invention is to provide a DNA construct capable of expressing a desired transgene in a host cell in a trackable manner. As illustrated by FIGS. 1A-1C, such a DNA construct operably links a fluorescent reporter positioned within a spliceable intron to a desired gene, wherein expression of both genes is regulated by a single promoter and transcription terminator. The fluorescent reporter and the desired gene are transcribed together as a single pre-mRNA that can further proceed to non-spliced or spliced mRNA, wherein the non-spliced mRNA is translated into the fluorescent protein, while the spliced mRNA produces the desired product. In this way, the presence of the fluorescent reporter protein indicates expression of the desired gene.

Another aim of the present invention is to provide a method of producing transgenic host cells having a desired product. Upon entry into the host cell or organism population, the DNA construct provided by the present invention is capable of expressing both fluorescent reporter and desired product in the same host cell, in particular, all cells having a phenotype of the fluorescent reporter also express the desired gene. Therefore, a transgenic host cell having a desired product is easily identified by visualizing the fluorescent signal. Such a method is rapid and convenient to perform.

### BRIEF DESCRIPTION OF THE DRAWINGS

FIGS. 1A-1C illustrate schematically the invention. FIG. 1A is a general representation of the DNA construct according to the invention. FIG. 1B is the non-spliced mRNA product produced by FIG. 1A, which translates to the fluorescent reporter. FIG. 1C is the spliced mRNA produced by FIG. 1A, which translates to the desired product.

FIGS. 2A-2B illustrate expression of pGR plasmid in transfected human embryonic kidney (HEK) 293T cells used for Example 1. FIG. 2A depicts the pGR plasmid used in Example 1. CMV promoter is the human cytomegalovirus promoter that initiates gene transcription. SV40polyA is the SV40 late polyadenylation signal that terminates gene transcription. AmpR is DNA segment conferring resistant phenotype to ampicillin. pUCori is pUC origin for replication of the plasmid in bacteria. FIG. 2B is the expression results of pGR vector as visualized by the fluorescent microscope.

FIGS. 3A-3B illustrate expression of pRE plasmid in transfected human embryonic kidney (HEK) 293T cells used for Example 2. FIG. 3A depicts the plasmid used in Example 2. FIG. 3B depicts the expression results of pRE vector as visualized by the fluorescent microscope.

FIGS. 4A-4D illustrate expression of pIGR-EGFP lentiviral vector in human embryonic kidney (HEK293) cells used in Example 3. FIG. 4A depicts the pIGR-RGFP lentiviral vector used in Example 3. 5′-LTR is 5′-long-terminal repeats of virus. Psi is a packaging signal of virus. RRE is a Rev response element of virus. CTS is a DNA segment derived from the human immunodeficiency virus type 1 pol gene. Ubc promoter is the human Ubiquitin C promoter. RtTA3-Sh ble fusion gene is a DNA segment consisting of a reverse transactivator and Zeocin-resistant gene. WPRE is Woodchuck Hepatitis Virus Posttranscriptional Regulatory Element. 3′-LTR is 3′-long-terminal repeats of virus. RBG polyA is a rabbit globin polyadenylation signal. SV40ori is replication origin of simian virus 40. On is pBR322 replication origin. FIG. 4B is the gene expression results of Example 3 in the absence or presence of 0.5 ug/ml Doxycycline as visualized by fluorescent microscope. FIG. 4C is flow cytometric analysis of expression of turboRFP and EGFP. FIG. 4D shows spliced and non-spliced RNA products upon Doxycycline induction as detected by the reverse-transcription polymerase chain reaction (RT-PCR).

FIGS. 5A-5C depict inducible expression of pIGR-GAPDH/V5 vector in HEK293 cells used in Example 4. FIG. 5A depicts the pIGR-GAPDH/V5 lentiviral vector used in Example 4. FIG. 5B shows expression of turboRFP in three cell clones in the absence or presence of 0.5 ug/ml Doxycycline as visualized by fluorescent microscope. FIG. 5C shows expression of GAPDH/V5 in three cell clones of FIG. 5B as determined by Western-blot.

FIGS. 6A-6B depict inducible expression of pIGE-GAPDH/V5 vector in chinese hamster ovary (CHO-K1) cells used in Example 5. FIG. 6A depicts the pIGE-GAPDH/V5 lentiviral vector used in Example 5.

FIG. 6B shows expression of EGFP in three cell clones in the absence or presence of 0.5 ug/ml Doxycycline as visualized by fluorescent microscope. FIG. 6C shows expression of GAPDH/V5 in three cell clones of FIG. 6B as determined by Western-blot.

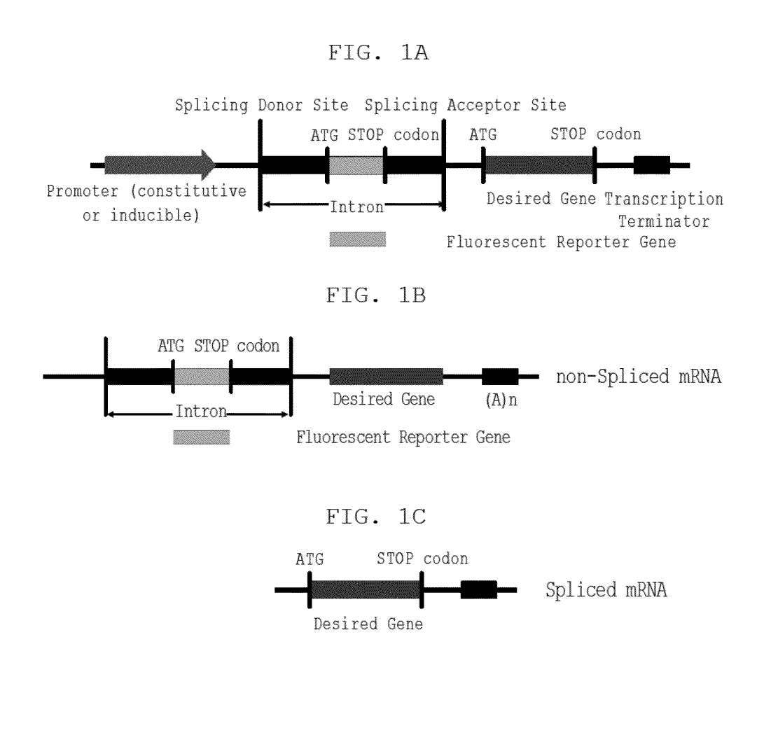

### DETAILED DESCRIPTION OF THE INVENTION

In a preferred embodiment of the invention, a recombinant DNA construct is provided for expression of the desired gene in transgenic host cells or organism population. Such a construct comprises in order from 5′ to 3′: a promoter, a fluorescent reporter positioned within an intron, a desired gene, and transcription terminator.

The term “DNA construct” as used herein refers to an artificially constructed segment of nucleic acid. It can be an isolate or integrated in another DNA molecule. Accordingly, a “recombinant DNA construct” is produced by laboratory methods. The term “promoter” as used herein refers to a nucleotide sequence that enables a gene to be transcribed. Examples for mammalian promoters for use in the invention include, the mammalian cytomegalovirus or CMV promoter, the translational elongation factor EF-1a promoter, the SV40 promoter, the actin promoter, the glyceraldehyde 3-phosphate dehydrogenase (GAPDH) promoter, the ubiquitin promoter, the heat shock protein 70 (HSP70) promoter, the Thymidine kinase (TK) promoter, the phosphoglycerate kinase (PGK-1) promoter, or any promoter known in the art suitable for expression in mammalian cells. Yeast promoters for use in the invention include, for example, the alcohol dehydrogenase (ADH) promoter, the alcohol oxidase (AOX) promoters, the GAL1 promoter, the CYC1 promoter, the TEF promoter, the yeast PGK1 promoter, or any promoter known in the art suitable for expression in yeast cells. Baculoviral promoters for use in the invention include, for example, the polyhedron promoter, the p10 promoter from the AcNPV virus, the OpIE2 promoter, or any promoter known in the art suitable for expression in insect cells.

The term “fluorescent reporter gene” used herein refers to a DNA segment encoding a family of proteins that are capable of forming a visible wavelength chromophore. A fluorescent reporter protein is the protein product encoded by a fluorescent reporter gene. Upon introducing a DNA construct having a fluorescent reporter gene into a living cell, the location and dynamics of the fluorescent protein can be visualized by a fluorescence microscopy. Any fluorescent reporter gene is useful for the invention. Examples of fluorescent reporter proteins include, EGFP, turboRFP, TagBFP, mTagBFP2, EBFP2, mKalamal, mCherry, ECFP, mTurquoise2, mTurquoise, TagCFP, mTFP1, TagGFP2, EYFP, SYFP2, TagYFP, mOrange, mOrange2, tdTomato, mRaspberry, mStrawberry, mTangerine, TagRFP, TagRFP-T, mRuby, mPlum, or any fluorescent protein suitable for expression in eukaryotic cells.

The term “intron” as used herein refers to both the DNA sequence within a gene and the corresponding sequence present in the pre-mRNA, in which nucleotide sequence can be excised, or removed from the pre-mRNA by the splicing machinery provided by a cell host. Accordingly, “splice” is the process of excision of an intron sequence present in a pre-mRNA molecule. Typically introns comprise a short nucleotide sequence that is highly conserved at the boundary between an exon and an intron, referred to as the 5′ splice site (also called splice donor site or “SD”); a branch sequence consisting of 20-50 nucleotides upstream of acceptor site; the boundary between an intron and an exon, referred to as the 3′ splice site (also called acceptor site or “SA”) that is highly conserved, preceded by a pyrimidine-rich region.

In a given eukaryotic host cell, an intron can be either spliced or non-spliced from the pre-mRNA. Consequently, a single pre-mRNA is capable of generating a heterogeneous pool of mature mRNA transcripts in a transgenic cell. Accordingly, a “spliceable intron” is an intron sequence that is capable of being spliced efficiently from the pre-mRNA by the machinery provided by a cell host. Typically, the spliced mRNA is translated to the desired protein, while the non-spliced mRNA produces the fluorescent reporter protein.

An intron is modified to contain a fluorescent reporter gene not normally present within the intron using the known recombinant DNA methods. Typically, a fluorescent reporter gene is inserted into an intron by cleaving the intron with a restriction endonuclease, and then ligating the resulting DNA fragment to the fluorescent reporter gene with a DNA ligase. Another method is to use polymerase chain reaction (PCR) to insert the fluorescent reporter into an intron.

The efficiency of intron splicing highly depends on the sequences of splice donor and acceptor sites. In general, naturally occurring introns are capable of being efficiently spliced. These introns and splice sites have been reported by abundant literatures and can be immediately used by the skilled person (Mount, Nucleic Acids Res. 10:459-472, 1982; Enapathy, et al., Methods Enzymol., 183:252-278, 1990). Artificially designed introns are also useful (Winey et al., Mol. Cell Biol., 9:329, 1989; Gatermann et al, Mol. Cell Biol., 9: 1526, 1989). Examples of introns used in the invention include, the intron A from human cytomegalovirus (Towne) immediate-early gene, the first intron from eukaryotic translational elongation factor or EF-1alpha, the first intron from eukaryotic beta-globin, the first intron from mammalian beta-actin, the first intron from mammalian ubiquitin, an intron from immunoglobulin, a chimeric intron consisting of 5′-splice donor site from the first intron of the human β-globin gene and 3′-splice acceptor site from the intron of an immunoglobulin gene, a 5′-UTR intron from yeast Ribosomal protein S8A, a 5′-UTR intron from yeast Ribosomal protein L32, a 5′-UTR intron from yeast Ribosomal protein S29, or any intron that can be efficiently spliced in eukaryotic cells.

The intron used for the present invention should be spliceable. The splicing efficiency determines the ratio of spliced and non-spliced mature mRNA, and hence protein products of the fluorescent reporter and the desired gene. While the naturally occurring intron sequences are usually efficient for splicing, the efficiency of intron splice may also be adjusted by modifying the sequence of splice acceptor sites (Fallot, S. et al. Nucleic Acids Research 37: e134, 2009; and U.S. Pat. No. 0,305,343 A1, 2009).

The term “desired gene” as used herein refers to a DNA segment encoding a protein of interest. Accordingly, a “desired product” is a protein encoded by a desired gene.

Any desired gene that is suitable for expressing in a host cell can be used for the invention. Examples of desired genes include those encoding, a toxin, such as ricin holotoxin or its subunits, pseudomonas exotoxin (PE), diphtheria toxin (DT), gelonin, and alpha-sarcin; a tumor suppressor, such as retinoblastoma (RB) protein, p53 tumor-suppressor protein (TP53), PTEN, VHL, APC, CD95, ST5, YPEL3, ST7, and ST14; an oncoprotein, such as RAS, WNT, MYC, ERK, TRK, myc, and BCR-Abl; tissue-type plasminogen activator (tPA); a human serum albumin and bovine serum albumin; blood clotting factor VIII, IX, and thrombin; an anti-clotting factor, such as Protein C; a nuclease, such as DNase and RNase; a growth factor, such as vascular endothelial growth factor (VEGF), epidermal growth factor (EGF), insulin-like growth factor (IGF), transforming growth factor (TGF-alpha and TGF-beta), fibroblast growth factor (FGF), platelet-derived growth factor (PDGF), nerve growth factor (NGF), erythropoietin (EPO), granulocyte colony-stimulating factor (G-CSF) and granulocyte-macrophage colony-stimulating factor (GM-CSF); an immunoglobulin, such as IgG, IgA, IgE, IgM, single-chain Fv, and Fab antibody; a protein used for antibody affinity purification, such as Protein A, Protein G, and Protein L; an enzyme, such as alkaline phosphatase, horseradish peroxidase, beta-galactosidase, ATPase, GTPase, protein kinase A, protein kinase C, casein kinase 2, mitogen-activated protein kinases (MAPKs), AKT kinase, Ca2+/calmodulin-dependent protein kinases or CaM kinases, phosphorylase kinase (PhK), PTP1B, PP2C, PTEN, CDC25A, CDC25B, CDC25C, a histone acetyltransferase; a deacetylase; a methyltransferase; a growth hormone, such as HGH and BGH; growth factor releasing hormone; neurotrophin-3, -4, -5, and -6; a cytokine, such as tumor necrosis factors (TNF-alpha and TNF-beta, interferon-alpha, beta, and gamma, interleukin-2, 6, 8, and IL-12; a CD molecule, such as CD3, CD4, CD8, CD19, CD20, CD22, CD30, CD45, CD54, CD74, and CD137; an epidermal growth factor receptor, such as EGFR, ErbB2, ErbB3, and ErbB4; a G protein-coupled receptor, such as beta-adrenergic receptors, prostaglandin E2 receptors, rhodopsin, CCRL2, GPR1, GPR3, GPR4, GPR17, GPR75, P2RY10, OXGR1, SUCNR1, LGR4, LGR5, MRGPRX2, OPN3, OPN5, P2RY8, BAI1, CELSR1, EMR1, LPHN1, LPHN2, LPHN3, T1R1, T1R2, and T1R3; a nuclear receptor, such as estrogen receptor (ER), glucocorticoid receptor (GR), androgen receptor (AR), and progesterone receptor (PR); a chromatin remodeling protein, such as PBAF, ARID1A, ACTL6A, ACTL6B, BRG1 and BRM; a translational elongation factor, such as EF1A1, EF1A2, EF1B, EF1D, EF1E1, EF1G, and EF2; glyceraldehyde 3-phosphate dehydrogenase (GAPDH); a fluorescent protein, such GFP, turboRFP, and mCherry; protein chimeras, such as immunoadhesins, a growth hormone-toxin fusion protein, a bispecific antibody composed of two scFv antibodies targeting different antigens, a growth factor-toxin fusion protein, an antibody-toxin fusion protein, or any fusion proteins known in the art suitable for expression in eukaryotic cells.

A desired gene is isolated from a host cell or organism population by using any of the known methods for isolating a polynucleotide. Alternatively, a desired gene is obtained using the known methods for DNA synthesis.

Typically, for the convenience of incorporating a desired gene into an expression vector, a short segment of DNA called “polylinker” that contains multiple restriction sites is often synthesized and inserts into the vector. The polylinker is cleaved with one or more restriction endonucleases and then ligates the resulting DNA fragment to the desired gene with a DNA ligase.

The term “transcription terminator” as used herein refers to a DNA sequence normally located at 3′-terminus of a DNA construct or genomic DNA, which causes termination of transcription. In eukaryotes, transcription terminators are recognized by protein factors and termination is followed by polyadenylation, a process of adding a poly(A)n tail to the mRNA transcripts in presence of the poly(A) polymerase. Transcription terminators for use in the invention include, for example, the human growth hormone (HGH) polyadenylation signal, the SV40 late polyadenylation signal, the rabbit beta-globin polyadenylation signal, the bovine growth hormone (BGH) polyadenylation signal, an AOX1 transcription termination sequence, a CYC1 transcription termination sequence, or any transcription termination sequence known in the art suitable for regulating gene expression in eukaryotic cells.

The DNA construct of the invention can be incorporated into a polynucleotide called “plasmid” that may further comprises additional sequences for the entry of the vector into cells, the replication of the vector in cells, the selection of transgene in cells, and other functional elements. Such elements can be isolated from commercially available vectors, such as pUC18, pUC19, pUC57, pcDNA3, pCI, pEF4/myc-His, phCMV1, p427-TEF, pPICZ, pPIC9, pBacPAK8, pBacPAK9 and pFastBac Dual using standard molecular biology techniques. Alternatively, these elements can be synthesized using known methods suitable for DNA synthesis.

The DNA construct of the invention can be incorporated into a viral vector that may further comprises additional sequences for packaging the viral particles, the propagation of the vector in cells, the entry of the vector into cells, the integration into chromosomes, and any other functional elements. While in a viral vector, the transcription terminator of the DNA construct of the invention may be removed for producing virus with high titers, remaining such a sequence results in expression of the desired gene at higher levels. Examples of commonly used viral vectors include a lentiviral vector, an adeno-associated viral vector, an adenoviral vector, a retroviral vector, a baculoviral vector, or any viral vector known in the art.

The term “recombinant virus” as used herein refers to a virus particle produced by recombinant DNA technology. Typically, production of recombinant virus involves inserting into an appropriate host cell a viral vector having a desired gene together with other polynucleotides called “packaging” plasmids. Examples for packaging plasmid include pMD2.G and psPAX2 for lentivirus, pCMV-VSV-G and pCMV-Gag-Pol for retrovirus, and any other plasmids suitable for producing a recombinant virus. After culturing, viruses having the desired gene are produced by the host cell and secreted into the culture medium.

Accordingly, a recombinant viral vector having a DNA construct of the invention can be used to produce viral particles. This is performed by co-transfecting a population of host cells called “packaging cell” the recombinant viral vector together with packaging plasmids. After culturing in a given medium, the virus particle is harvested from the growing culture medium.

Various methods and reagents can introduce a polynucleotide or viral vector into a target eukaryotic cell. Examples of such a reagent include a Lipofectamine reagent (supplied by Life Technologies), polyethylenimine (supplied by Sigma), calcium phosphate (supplied by Promega), and an electroporation reagent (supplied by Lonza). In addition, recombinant viral particles are useful for carrying a DNA construct into a eukaryotic cell, in particular a non-dividing cells. Commonly used recombinant viral particles include lentivirus, retrovirus, and adenovirus.

In an embodiment of the invention, a DNA construct described previously is for use in expressing the desired gene in a constitutive manner, wherein the desired gene is transcribed continually.

In one embodiment of the invention, a polynucleotide described previously is for use in expressing the desired gene in an inducible manner, wherein the desired gene is transcribed, as opposite to constitutive expression, only in the presence of a particular molecule called “inducer”.

While constitutive gene expression is achieved by using the invention with no need for further modification, inducible expression of a desired gene is accomplished in a more complicated way. First, a DNA segment called “operator” is isolated and operably linked to the promoter at a proximal distance. Second, a DNA construct that constitutively produces a repressor protein is required. Using known methods in the art, DNA constructs of the desired gene and the repressor are inserted into a population of host cells. The transgenic host cell then produces the repressor protein that inhibits transcription of the desired gene through binding to the operator sequence. Third, in order to trigger the expression of the desired gene, an inducer is added to the cell to remove the repressor from binding of the operator.

An operator sequence is operably linked to the promoter of the invention using standard recombinant DNA techniques. Examples for operators for use in the invention include a tet operator, a lac operator, a trp operator, an ara operator, a CuO operator, or any operator known in the art suitable for inducible gene expression. Examples for repressor for use in the invention include a CymR repressor, a tetracycline repressor, a lac repressor, a VgEcR/RXR heterodimer, or any repressor known suitable for expression in eukaryotic cells.

Inducers can function by disabling repressors or binding to activators. In any case, binding of the inducer can start gene expression and removing the inducer stops transcription. An inducer is either a biological process or an artificial method. Typically, in an inducible gene expression system, an inducer is a chemical molecule. Examples for inducers for use in the invention include, tetracycline, doxycycline, ecdysone, isopropyl β-D-1-thiogalactopyranoside, arabinose, allolactose, muristerone A, cumate, trimethyl cumate, butyrate, or any inducers known in the art suitable for triggering gene expression in eukaryotic cells.

An embodiment of the invention relates to a method of producing a host cells comprising introducing a DNA construct described previously into a population of host cells.

Routine procedures for producing a host cell having a desired transgenic product are time-consuming and expensive. The method provided by the invention is particularly useful for rapid production of such a host cell, because the presence of a fluorescent reporter precisely indicates the expression of the desired product. A skilled person can introduce the DNA construct described previously into a population of host cells. After culturing in a given medium, a host cell having a phenotype of the fluorescent reporter is easily identified by a fluorescent microscope or a flow cytometer that needs limited numbers of cells. Therefore, such a method eliminates the need for expanding each cell clone to a large population for identifying a positive one.

The method provided by the invention is useful for producing a host cell that is capable of expressing a desired product in an inducible manner. As described previously, the promoter of the DNA construct described previously is able to operably link to an operator sequence, resulting in an inducible expression cassette that produces a desired transgenic product only in the presence of an inducer. Together with a second DNA construct having a repressor, the inducible expression cassette is introduced into a population of host cells. After culturing in a given medium, an inducer is added to the cells. An inducible transgenic host cell is identified by the phenotype of the fluorescent reporter using a fluorescent microscope or a flow cytometer, which is performed on a limited number of cells. Hence, the method provided by the invention is able to eliminate the need for expanding each cell clone to a large population for identifying an inducible one.

Examples of eukaryotic host cells suitable for use in the invention include mammalian cells, insect cells and yeast cells. Typically, such cells are Chinese hamster ovary (CHO) cells; human embryonic kidney (HEK) cells; PER.C6 cells; BHK-21 cells; NIH3T3 cells; myeloma cells, such as mouse NS0 and Sp2/0; COS cells; liver cancer cells, such as HepG2, SNU-475, SK-HEP-1, and Hep3B; human lymphoma cells, such as Daudi, Raji, JM-1, and Namalwa; leukemia cells, such as HL-60, KU812, Kasumi-1, CCRF-CEM, KG-1, THP-1, and K-562; breast cancer cells, such as BT-474, SK-BR3, MDA-MB-361, MDA-MB-453, BT-20, MDA-MB-435, MCF-7, MDA-MB-231, ZR-75-1, and T-47D; lung cancer cells, such as NCI-H2122, NCI-358, NCI-H292, NCI-H522, NCI-H820, HCC-827, and NCI-H23; melanoma cells, such as CHL-1, HMCB, CHL-2, M101, SK-MEL-28, SK-MEL-5, and SK-MEL-1; glioma cells, such as LN-18, LN-229, A172, SW1088, U118-MG, and U87-MG; neuroblastoma cells, such as SK-N-BE(2), SK-N-AS, Neuro-2a, SK-N-SH, SK-M-NC, and N1E-115; colon cancer cells, such as HT29, HT115, HT55, Caco-2, LS180, SW620, LoVo, LS174T, COLO 205, and SW 1116; Spodoptera frugiperda Sf9 and Sf21 cells; High Five insect cells; Saccharomyces cells; Pichia pastoris cells and any cells suitable for producing a desired protein.

An embodiment of the invention relates to a method of producing a desired protein, which comprises culturing a host cell produced by the invention. The method further comprises isolating the protein from the culture.

### EXAMPLES

In the following description, all molecular biology experiments are performed according to standard protocols (Sambrook J, Fritsch E F and Maniatis T (eds) Molecular cloning, A laboratory Manual 2nd Ed, Cold Spring Harbor Laboratory Press). All restriction digestion enzymes, Klenow fragment and T4 DNA ligase are purchased from New England Biolabs Inc. Cloned pfu DNA polymerase is from Clontech.

### Example 1

### An Intronic Enhanced Green Fluorescent Reporter (EGFP) is Capable of Indicating Expression of Desired Product

In order to determine if a fluorescent reporter positioned within an intron can be expressed efficiently and be used to indicate expression of a desired gene, the enhanced green fluorescent reporter gene (EGFP) is inserted into an intron, while a red fluorescent protein (turboRFP) gene is positioned at 3′-terminus of the intron splicing acceptor site as a representation of desired genes. The transcription of both genes is regulated by a single promoter and a unique transcription terminator in a constitutive way. After introducing the vector into mammalian cells, fluorescent EGFP and turboRFP are expressed and analyzed by a fluorescent microscope.

### 1. Vector Construction:

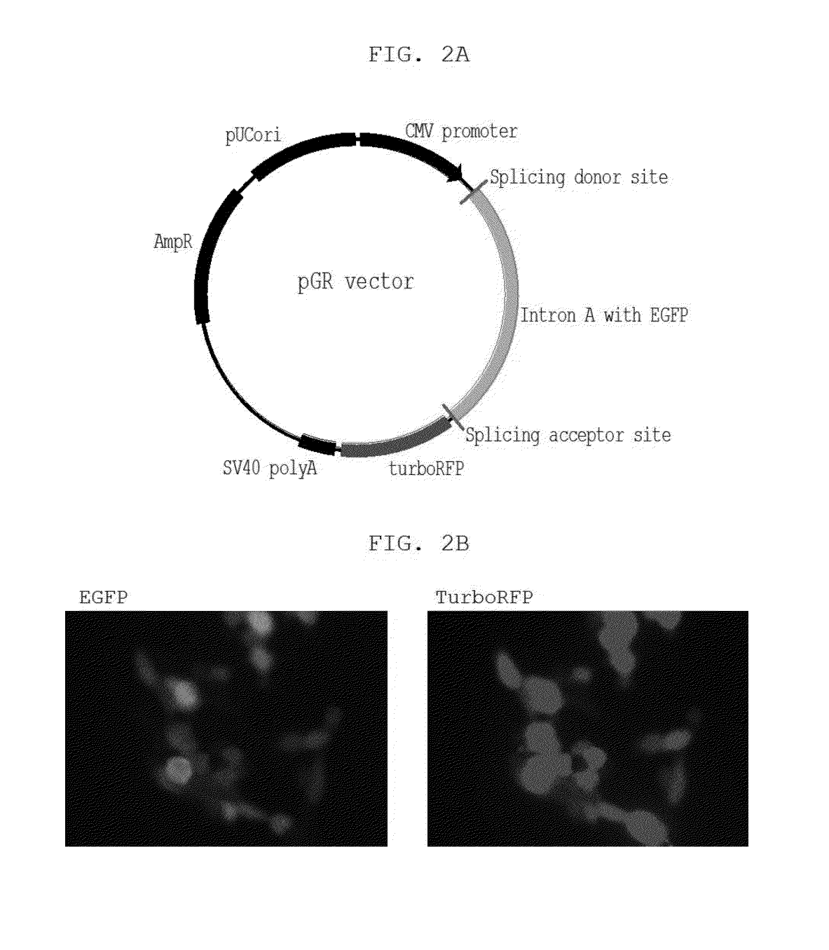

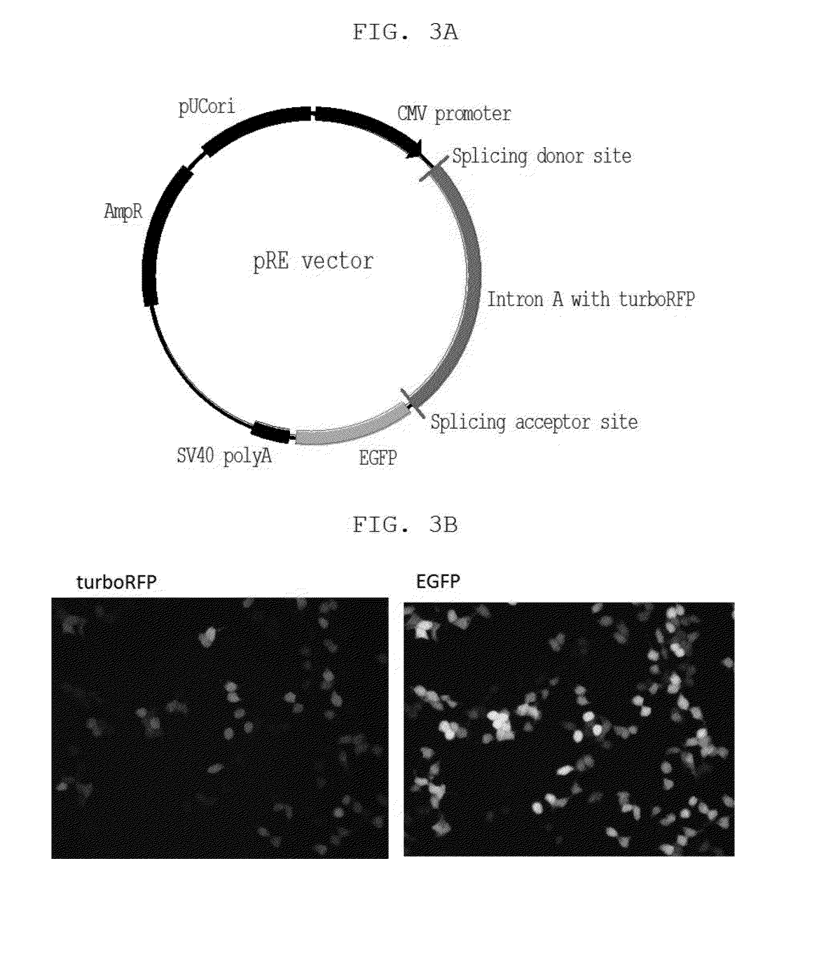

The vector for constitutive gene expression is illustrated by FIG. 2A. A single human cytomegalovirus (CMV) promoter regulates expression of turboRFP and EGFP in a constitutive way. The enhanced green fluorescent protein (EGFP) gene is positioned within the large intron A sequence from the human cytomegalovirus (Towne) immediate-early gene. The turboRFP gene located at 3′-end of the splice acceptor site of the intron A.

A basic pCIA vector was constructed by excising the human CMV promoter from the gWIZ-blank vector (Genlantis) with MscI/SalI restriction enzymes and ligating into the pCI vector (Promega) cut by MscI/XhoI. Briefly, 2 ug of gWIZ-blank were digested by MscI and SalI for 2 hours at 37° C. The 1627 bp DNA fragment was purified from 1% agarose gel by QIAquick Gel Extraction Kit. 1 ug of pCI vector was digested by MscI and XhoI for 2 hours at 37° C. The 2958 bp DNA fragment was purified from 1% agarose gel by QIAquick Gel Extraction Kit. 50 ng of the 2958 bp DNA fragment was mixed with 83 ng of the 1627 bp DNA fragment. 1 uL of T4 DNA ligase was added to the DNA mixture and incubated for one hour at 25° C. 2 uL of the ligation were added to 100 uL of competent DH5alpha bacterial cells and incubated on ice for 10 minutes. After incubating for 45 seconds at 42° C., 250 uL SOC medium (Life Technologies) were added. Cells were further cultured for one hour at 37° C. with shaking at 250 rpm. 100-microliter of the culture were grown on a LB-agar plate having 50 ug/ml ampicillin (Research Products International Corporation). After culturing for 16 hours at 37° C., single bacterial colonies were isolated from the plate and cultured in 3 ml LB medium having 50 ug/ml ampicillin for 20 hours at 37° C. with shaking at 250 rpm. Bacterial cells were harvested by centrifugation for 10 minutes at 2,000 rpm. Plasmids were isolated from the cell pellets by QIAprep Spin Miniprep Kit (Qiagen). The plasmids were analyzed by BglII/BamHI double digestion to confirm the recombinant pCIA vector.

The intron A was partially amplified from the pCIA vector. Primers used for intron A amplification are,

Intron A forward primer sequence:
(SEQ ID NO: 1)
5′-GCCATCCACGCTGTTTTG-3′
Intron A reverse primer sequence:
(SEQ ID NO: 2)
5′-CTGACCATGGTGTCCGGATGGATAAGAGCCAAAGGGGTGTGCC-3′

The full-length EGFP gene was amplified from the pEGFP-1 vector by polymerase chain reaction (PCR). The reverse primer also contained a SphI restriction digestion sequence for inserting into the pCIA vector.

EGFP forward primer sequence:
(SEQ ID NO: 3)
5′-CCATCCGGACACCATGGTCAGCAAGGGCGAGGAGCTGTTC-3′
EGFP reverse primer sequence:
(SEQ ID NO: 4)
5′-CTGACGTGCATGCGTCACTTGTACAGCTCGTC-3′

The PCR products were then assembled together using splice overlap extension PCR.

The assembled DNA segment was digested by SacII/SphI restriction enzymes and ligated into the pCIA vector cut by the same enzymes. 2 uL of the ligation were transformed into 100 uL of competent DH5alpha cells. 100 uL of the culture were grown on a LB-agar plate having 50 ug/ml ampicillin. After culturing for 16 hours at 37° C., single bacterial colonies were isolated from the plate and cultured in 3 ml LB medium having 50 ug/ml ampicillin for 20 hours at 37° C. with shaking at 250 rpm. Bacterial cells were harvested by centrifugation for 10 minutes at 2,000 rpm. Plasmids were isolated by QIAprep Spin Miniprep Kit. The resulted pIGFP plasmids were analyzed by DNA sequencing using the primer SEQ ID NO: 1.

The gene encoding turboRFP was amplified from the pTRIPZ vector (Thermoscientific) by PCR and inserted into the pIGFP vector. The PCR primers also contained restriction digestion sequences for inserting into the pIGFP vector (i.e. EcoRI HF site in 5′ position and SalI HF site in 3′ position). The primers for amplification of turboRFP are,

TurboRFP forward primer sequence:
(SEQ ID NO: 5)
5′-GATATCGAATTCCACCATGAGCGAGCTG-3′
TurboRFP reverse primer sequence:
(SEQ ID NO: 6)
5′-CTAATGGTCGACGATTATCTGTGCCCCAG-3′

The PCR products were digested by EcoRI HF/SalI HF and ligated into the pIGFP vector cut by the same enzymes. 2 uL of the ligation were transformed into 100 uL of competent DH5alpha cells. 100 uL of the culture were grown on a LB-agar plate having 50 ug/ml ampicillin. After culturing for 16 hours at 37° C., single bacterial colonies were isolated from the plate and cultured in 3 ml LB medium, containing 50 ug/ml ampicillin for 20 hours at 37° C. with shaking at 250 rpm. Bacterial cells were harvested by centrifugation for 10 minutes at 2,000 rpm. Plasmids were isolated from the cell pellets by QIAprep Spin Miniprep Kit. The resulted pGR plasmid was analyzed by DNA sequencing using the primer,

Sequencing primer sequence (SEQ ID NO: 7):
5′-CGCGCCACCAGACATAATAG-3′

The sequence verified pGR plasmid was used for the expression study.
2. Transfection of the pGR Plasmid into Human Embryonic Kidney (HEK) 293T Cells

The pGR plasmid was transfected into 293T cells (American Type Culture Collection or ATCC) using Lipofectamine LTX reagent (Life Technologies). Briefly, 24 hours prior to transfection, 2.0×10⁵ 293 T cells in Dulbecco's Modified Eagle's Medium (DMEM) having fetal bovine serum (Life Technologies) were added onto a 12-well cell culture plate. 1.25 ug of pGR plasmid was transfected to the cells using Lipofectamine LTX transfection reagent according to the manufacturer's instructions. Sixteen hours after transfection, the culture medium was removed. Cells were suspended in 3 ml fresh DMEM having 10% fetal bovine serum and transferred to a 6-cm culture dish. After 24 hours, expression of the EGFP reporter and desired turboRFP product were visualized under microscope.

As shown by FIG. 2B, transfected cells were capable of producing the EGFP reporter (green color), while all cells having the phenotype of EGFP also showed bright signal of turboRFP (red color), indicating tight correlation between the reporter and the desired product.

### Example 2

### An Intronic TurboRFP Fluorescent Reporter is Capable of Indicating Expression of Desired Product

In order to prove the possibility of other intronic fluorescent reporter as an indicator of expression of desired products, the turboRFP gene was inserted into an intron as a reporter, while the EGFP gene was positioned at 3′-terminus of the intron as a representation of desired genes. The transcription of both genes is regulated by a single promoter and a unique transcription terminator in a constitutive way. After introducing the vector into mammalian cells, fluorescent turboRFP and EGFP are expressed and analyzed by a fluorescent microscope.

### 1. Vector Construction:

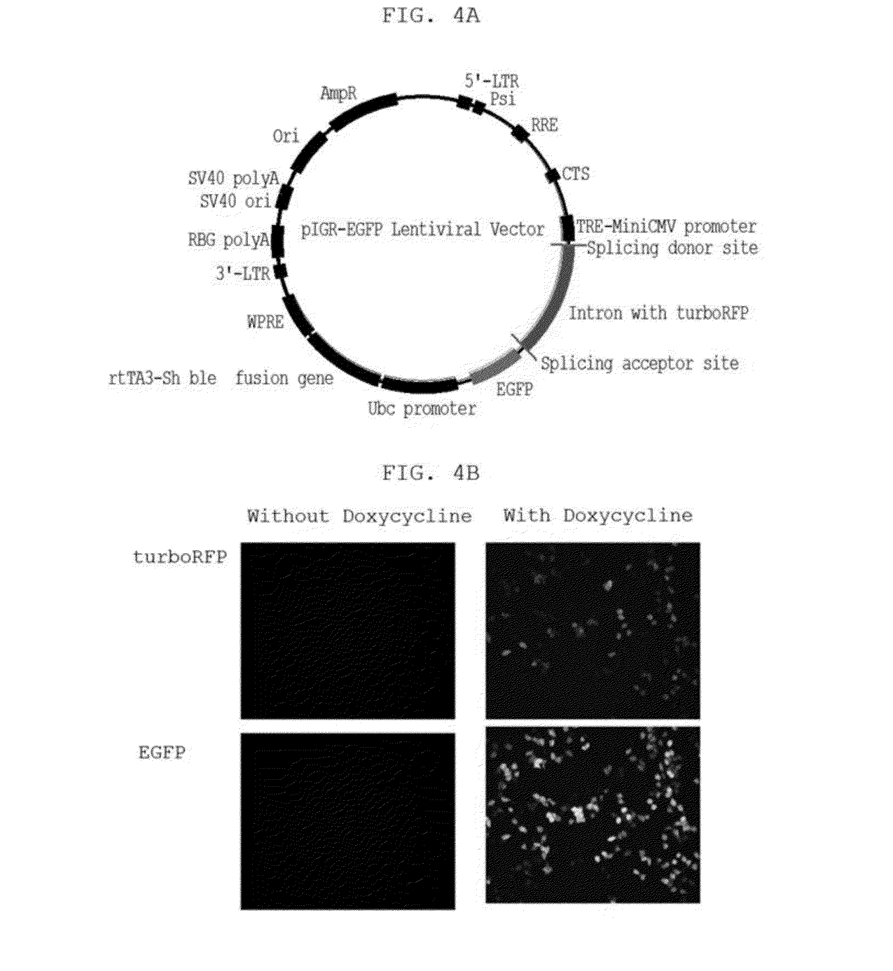

The vector for constitutive gene expression is illustrated by FIG. 3A. Expression of turboRFP and EGFP are regulated by the CMV promoter. The gene encoding turboRFP is positioned within the intron A sequence from the human cytomegalovirus immediate-early gene. The gene encoding EGFP was placed at 3′-end of the splice acceptor site of the intron A.

The full-length turboRFP gene was amplified from the pTRIPZ vector by PCR. The PCR primers also included restriction digestion sequences for inserting into the pCIA vector (i.e. NsiI site in 5′ position and SphI site in 3′ position).

TurboRFP forward primer sequence:
(SEQ ID NO: 8)
CAGAATTCATGCATCACCATGAGCGAGCTGATCAAG
TurboRFP reverse primer sequence:
(SEQ ID NO: 9)
ATGATATCGCATGCGATTATCTGTGCCCCAGTTTGC

The PCR product was purified from 1.5% agarose gel and digested by NsiI/SphI. A 773 bp intron A fragment was produced by excising the pCIA vector with SphI/PstI. The digested PCR product and the intron A fragment were further inserted into the 3810 bp DNA fragment of the pCIA vector produced by NsiI/PstI by three fragments ligation. The resulting pIRFP vector was verified by DNA sequence analysis using the primer SEQ ID NO: 1.

The EGFP gene was isolated by PCR from the pEGFP-1 vector (Clontech). The PCR primers also contained restriction sites used for further insertion in the pIRFP plasmid (i.e. EcoRI site in 5′ position and MluI site in 3′ position).

EGFP forward primer sequence
(SEQ ID NO: 10)
CATCATGAATTCGCCACCATGGTGAGCAAG
EGFP reverse primer sequence
(SEQ ID NO: 11)
CTGATATCACGCGTTACTTGTACAGCTCGTCCATG

The EGFP PCR product was digested by EcoRI/MluI and inserted into the pIRFP vector excised by the same restriction sites. The resulting pRE plasmid was verified by DNA sequence analysis using the primer SEQ ID NO: 7.

2. Transfection of the pRE Plasmid into Human Embryonic Kidney (HEK) 293T Cells

The pRE plasmid was transfected into 293T cells using the Lipofectamine LTX reagent (Life Technologies). Briefly, 24 hours prior to transfection, 2.0×10⁵ 293 T cells in 2 ml of DMEM having fetal bovine serum (Life Technologies) were added onto a 12-well cell culture plate. 1.25 ug pRE plasmid was transfected to the cells using Lipofectamine LTX transfection reagent according to the manufacturer's instructions. Sixteen hours after transfection, the culture medium was removed. Cells were suspended in 3 ml fresh DMEM having 10% fetal bovine serum and transferred to a 6-cm culture dish. After 24 hours, expression of the turboRFP reporter and desired EGFP product were visualized under microscope.

As shown by FIG. 3B, cells were capable of producing turboRFP (red color) reporter. In addition, all cells having the phenotype of turboRFP also produced EGFP. These results indicated tight correlation between intronic fluorescent reporter and expression of the desired product.

### Example 3

### Inducible Expression of Enhanced Green Fluorescent Protein (EGFP) in Human Embryonic Kidney (HEK293) Cells

In order to investigate intronic fluorescent proteins as reporters for inducible expression of a desired gene, a DNA construct of the invention is incorporated into an inducible expression vector. The resulted vector is transduced to a population of host cells. In the absence of an inducer, neither the fluorescent reporter nor the desired product is expressed by the host cell. In the presence of an inducer, the fluorescent reporter is expressed by the transgenic host cell. A host cell having a phenotype of the fluorescent reporter should also produce the desired product.

### 1. Vector Construction:

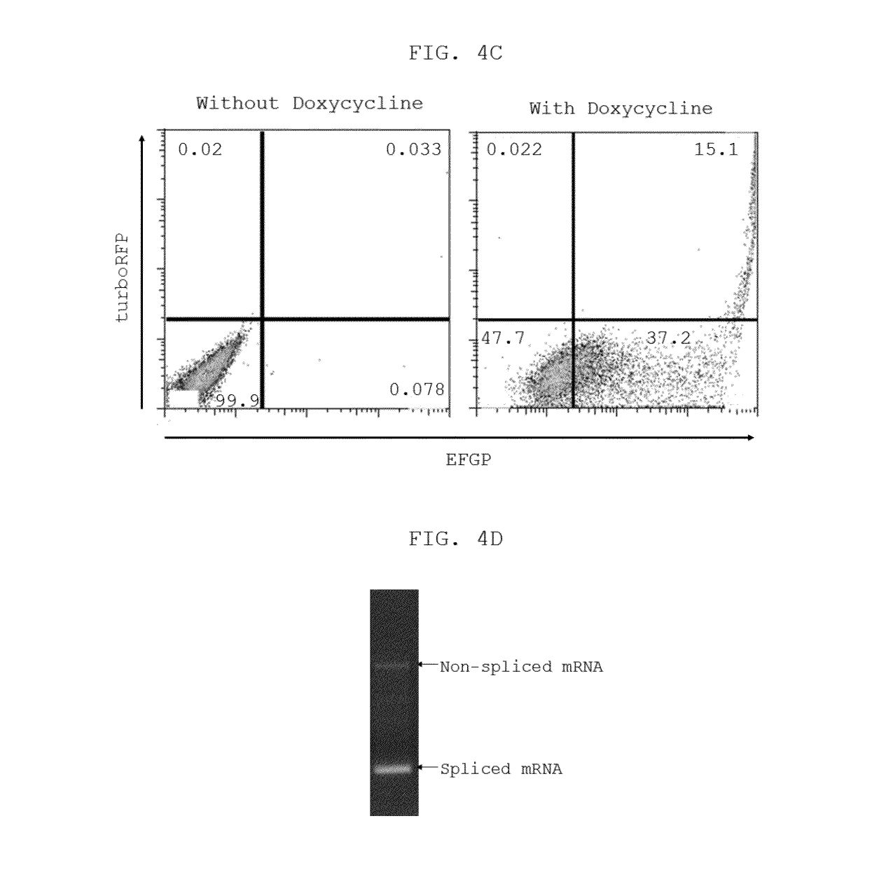

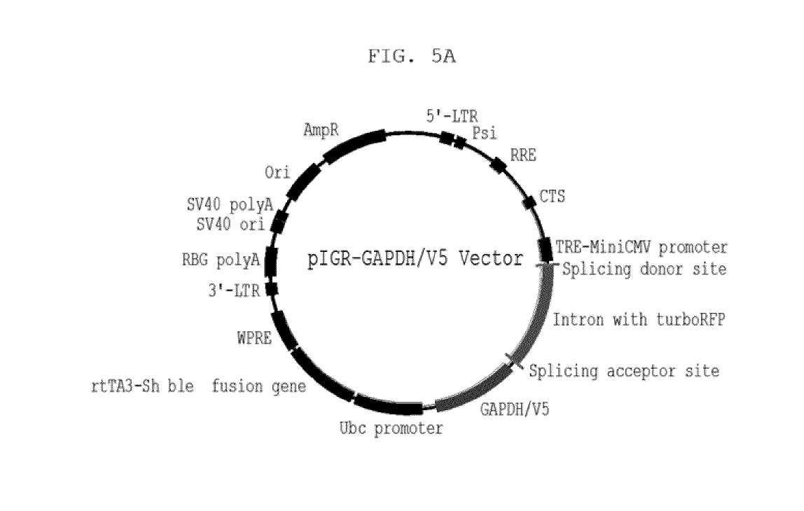

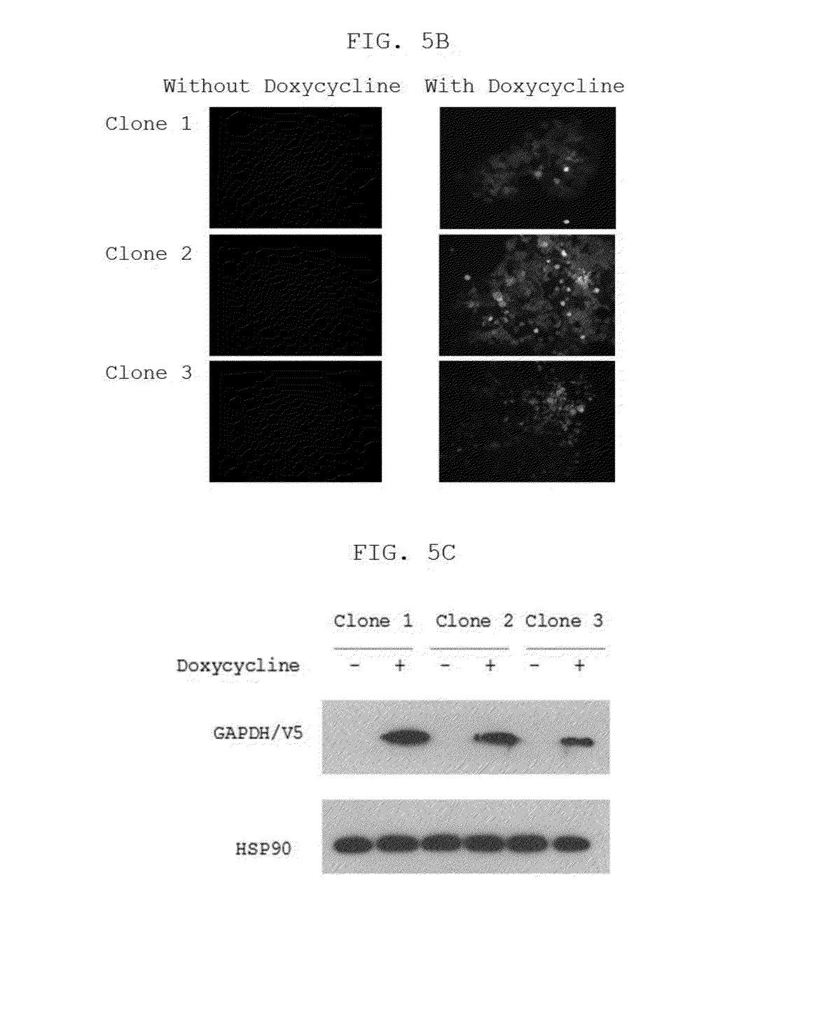

The lentiviral vector for inducible expression of the reporter and desired gene is illustrated by FIG. 4A. Expression of turboRFP and EGFP are inducible and regulated by a single operable linkage comprising the tetracycline response element (TRE) and a minimal CMV promoter. The turboRFP reporter gene is positioned within the intron A sequence from the human cytomegalovirus immediate-early gene. The EGFP gene located at 3′-end of the splice acceptor site of the intron A. In addition, the rtTA3 transactivator and sh ble genes are expressed as a fusion protein. The expression of the rtTA3-Sh ble fusion protein is regulated by the Ubc promoter and is constitutive.

A basic pTZ10 vector was constructed by inserting the 2437 bp DNA fragment of the psPAX2 plasmid (Addgene) excised by PvuI-HF/NheI (blunted) enzymes into the 9275 bp DNA fragment of pTRIPZ vector cut by PmeI/PvuI-HF. Briefly, 2 ug of the psPAX2 plasmid were digested by NheI for 2 hours followed by incubating with the Klenow fragment for 30 minutes at 37° C. The digestion reactions were further digested by PvuI-HF for 2 hours. The 2437 bp DNA fragment was purified from 1% agarose gel by QIAquick Gel Extraction Kit. 2 ug of pTRIPZ vector was double digested by PmeI and PvuI-HF for 2 hours. The 9275 bp fragment was purified from 1% agarose gel by QIAquick Gel Extraction Kit. 50 ng of the 9275 bp DNA fragment were ligated into 40 ng of 2437 bp fragment by T4 DNA ligase. 2 uL of the ligation were added to 100 uL of competent Stbl3 bacterial cells. 100 uL Transformed cells were grown on a LB-agar plate having 50 ug/ml ampicillin and 25 ug/ml Zeocin (InVivoGen). After culturing for 16 hours at 37° C., single bacterial colonies were isolated from the plate and cultured in 3 ml LB medium having 50 ug/ml ampicillin for 20 hours at 37° C. with shaking at 250 rpm. Bacterial cells were harvested. Plasmids were isolated by QIAprep Spin Miniprep Kit. The plasmids were digested by BamHI to confirm the recombinant pTZ10 vector.

The rtTA3 gene was amplified by PCR from the pTRIPZ vector. The sh ble gene was amplified by PCR from the pSecTag2A vector (Life Technologies). The PCR products were then assembled together using splice overlap extension PCR.

RtTA3 forward primer sequence:
(SEQ ID NO: 12)
GGCTTTTTTGTTAGACGCTAG
RtTA3 reverse primer sequence:
(SEQ ID NO: 13)
CTCCCTCTGGTCCTCCTGATGCAGCAGCCCCGGGGAGCATGTCAAGG
Sh ble forward primer sequence:
(SEQ ID NO: 14)
GCATCAGGAGGACCAGAGGGAGGTTCCATGGCCAAGTTGACCAG
Sh ble reverse primer sequence:
(SEQ ID NO: 15)
CATAATCAGCTGCGGCCGCGAAATCTCGTAGCACGTG

The assembled rtTA3-sh ble fusion gene product was purified from 1% agarose gel and digested by NheI/NotI. 2 ug of the pTZ10 vector was excised by NheI/NotI and the 9813 bp fragment was purified from 1% agarose gel. 20 ng of the digested PCR product was ligated into 50 ng of the 9813 bp fragment by the T4 DNA ligase. 2 uL of the ligations were transformed to 100 uL of competent Stbl3 bacterial cells. The transformed cells were grown on a LB-agar plate having 50 ug/ml ampicillin and 25 ug/ml Zeocin and cultured for 20 hours at 37° C. Single colonies were isolated from the plate and cultured in 3 ml LB medium having 50 ug/ml ampicillin for 24 hours at 37° C. Bacterial cells were harvested by centrifugation. Plasmids were isolated from the cell pellets by QIAprep Spin Miniprep Kit. The resulting pIK vector was verified by DNA sequencing analysis.

The intronic turboRFP sequence along with the EGFP insert was amplified by PCR using the pRE plasmid as a template. PCR primers also contained restriction sites used for further insertion into the pIK plasmid (i.e. AgeI site in 5′ position and MluI site in 3′ position).

Forward primer sequence:
(SEQ ID NO: 16)
5′-CTAATAACCGGICTGGAGACGCCATCCACG-3′
EGFP reverse primer sequence
(SEQ ID NO: 11)
5′-CTGATATCACGCGTTACTTGTACAGCTCGTCCATG-3′

The PCR productx were excised by AgeI/MluI and inserted into the pIK vector digested by the same restriction sites. The resulting pIGR-EGFP plasmid was verified by DNA sequence analysis.

### 2. Preparation of Lentiviral Particles:

Lentiviral particles were produced by 293T cells. Briefly, 24 hours prior to transfection, 5.0×10⁵ 293 T cells in 2 ml DMEM having 10% Tet-approved fetal bovine serum (Clontech) were added onto a 3.5-cm cell culture dish. Cells were transfected with 1.25 ug pIGR-EGFP, 0.94 ug psPAX, and 0.31 ug pMD2.G plasmids using the Lipofectamine LTX transfection reagent. Sixteen hours after transfection, cells were suspended in 5 ml DMEM having 10% Tet-approved fetal bovine serum and transferred to a 6-cm culture dish. After 48 hours, lentiviral particles in the culture medium were harvested and filtered through a 0.45□ filter. Lentiviral particles were stored at −80° C.

### 3. Stable Infection of HEK293 Cells:

5×10⁵ HEK293 cells (ATCC) were added onto a cell culture dish and cultured in an incubator having 5% CO₂. After 24 hours, 50 uL of lentiviral particles described previously and 8 ug/ml polybrene (Sigma) were added to cells and cultured overnight. Cells were suspended in 10 ml DMEM having 10% Tet-approved fetal bovine serum and transferred to a 10-cm cell culture dish. After 24 hours, 0.2 mg/ml Zeocin was added. The culture medium was replaced every 3 days. After 3 weeks, viable cells were split to 2 equal aliquots in 10-cm cell culture dishes. Cells were cultured for 24 hours to allow for attachment to the dishes.

### 4. Fluorescent Microscope Analysis:

Doxycycline (Clontech) at 0.5 ug/ml was added to one aliquot of cells. After culturing for 48 hours, cells were analyzed for the expression of turboRFP and EGFP using a fluorescent microscope (Nikon) and the results were shown in FIG. 4B. Cell aliquot without Doxycycline was used as control. In the absence of Doxycycline, neither turboRFP nor EGFP was expressed, while Doxycycline induced expression of turboRFP in a portion of Zeocin selected cells. Importantly, all cells having a phenotype of turboRFP also expressed EGFP. Therefore, the characteristic of the turboRFP reporter phenotype is capable of indicating the expression of EGFP in an inducible gene expression system.

### 5. Flow Cytometer Analysis:

Flow cytometer was used to investigate the expression of EGFP and turboRFP. As shown by FIG. 4C, in the absence of Doxycycline, neither turboRFP nor EGFP protein was detected in any Zeocin selected cell, indicating tight control of the expression. In the presence of Doxycycline, 15.1% of cells produced turboRFP. In addition, all reporter turboRFP-positive cells also expressed the desired EGFP product with no exception. These results clearly indicate that the invention is useful for an inducible expression system.

### 6. Analysis of RNA Intron Splice Pattern:

The cells treated by Doxycycline for 48 hours were harvested by centrifugation. Total RNA was extracted by RNeasy Plus Mini Kit (Qiagen). 2 ug of the total RNA were converted to single strand cDNA by High Capacity cDNA Reverse Transcription Kit (Life Technologies). Splice pattern of the RNA was analyzed by PCR against 50 ng cDNA. The forward PCR primer was designed to complement the sequence upstream the splice donor site, while the reverse primer complemented EGFP gene.

Forward primersequence:
(SEQ ID NO: 1)
5′-GCCATCCACGCTGTTTTG-3′
Reverse primer sequence:
(SEQ ID NO: 17)
5′-GTGAACAGCTCCTCGCCCTTG-3′

The result was shown in FIG. 4D. The PCR produced two products. After sequencing these PCR products, they were confirmed to derive from the spliced or non-spliced RNA transcript. As expected, the spliced RNA product was translated to EGFP, while the non-spliced RNA expressed turboRFP.

### Example 4

### Inducible Expression of the Transgenic Glyceraldehyde-3-Phosphate Dehydrogenase (GAPDH) Gene in Human Embryonic Kidney Cells (HEK293) Using Intronic TurboRFP as Indicator

### 1. Vector Construction:

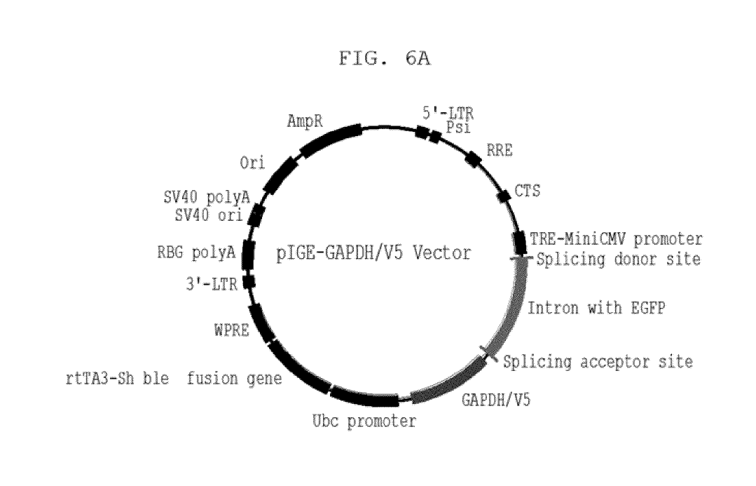

The lentiviral vector for inducible expression of the turboRFP reporter and desired GAPDH/V5 gene is illustrated by FIG. 5A. A V5-tag of the sequence “Gly-Lys-Pro-Ile-Pro-Asn-Pro-Leu-Leu-Gly-Leu-Asp-Ser-Thr” was added to the carboxyl-end of the human GAPDH gene for the detection of the protein.

The human GAPDH gene was cloned from 293T cells. Total RNA was isolated from one million of 293T cells by RNeasy Plus Mini Kit. 2 ug of the total RNA were converted to single strand cDNA by High Capacity cDNA Reverse Transcription Kit. The GAPDH gene was cloned by PCR against 50 ng cDNA. Primers designed to amplify the GAPDH gene also contained an EcoRI restriction site in 5′ position. A DNA sequence encoding the V5-tag was also added to the reverse primer and included a MluI restriction site in its 3′ position.

GAPDH forward primer sequence:
(SEQ ID NO: 18)
5′-CATACAGAATTCGACACCATGGGGAAGGTGAAG-3′
GAPDH reverse primer sequence:
(SEQ ID NO: 19)
5′-CTGATATCACGCGTCACGTAGAATCGAGTCCGAGTAGAGGGTTAG
GTATAGGCTTGCCCTCCTTGGAGGCCATGTGG-3′

PCR products were purified from 1.5% agarose gel and digested by EcoRI/MluI. The digested GAPDH/V5 PCR products were inserted into the larger fragment of pIGR-EGFP plasmid excised by the same restriction enzymes. 2 uL of the ligation were transformed to 100 ul of competent Stbl3 cells. The transformed cells were grown on a LB-agar plate, containing 50 ug/ml ampicillin and 25 ug/ml Zeocin for 16 hours at 37° C. Single colonies were isolated from the plate and cultured in LB medium having 50 ug/ml ampicillin for 20 hours at 37° C. with shaking at 250 rpm. Bacterial cells were harvested by centrifugation. Plasmids were isolated from the cell pellets by QIAprep Spin Miniprep Kit. The resulting pIGR-GAPDH/V5 vector was verified by DNA sequence analysis.

### 2. Preparation of Lentiviral Particles:

5×10⁵ of 293T cells in DMEM having 10% Tet-approved fetal bovine serum were added onto a 3.5-cm cell culture dish and cultured for 24 hours. Cells were co-transfected with 1.25 ug pIGR-GAPDH/V5, 0.94 ug psPAX, and 0.31 ug pMD2.G using the Lipofectamine LTX transfection reagent. Sixteen hours after transfection, culture medium was removed. Cells were suspended in 5 ml DMEM having 10% Tet-approved fetal bovine serum and transferred to a 6-cm culture dish. After 48 hours, the culture medium was removed from the cells and filtered through a 0.45 u filter. The filtered Lentiviral particles were stored at −80° C.

### 3. Stable Transfection of HEK293 Cells:

Twenty-four hours prior to infection, 5×10⁵ HEK293 cells were added onto a 3.5-cm cell culture dish. After 24 hours, 50 uL of lentiviral particles and 8 ug/ml polybrene were added to the cells and incubated for 16 hours. The culture medium was removed and cells were suspended in 10 ml of fresh DMEM having 10% Tet-approved fetal bovine serum. Cells were transferred to a 10-cm cell culture dish. After 24 hours, 0.2 mg/ml Zeocin was added. Culture medium was replaced every 3 days. After 3 weeks, ten viable cell clones were isolated and split to 2 equal aliquots in 96-well cell culture plates. The cells were grown for 24 hours to allow attachment to the plate.

### 4. Fluorescent Microscope Analysis:

Doxycycline at 0.5 ug/ml was added to one aliquot of isolated cell clones. After culturing for 48 hours, cells were analyzed for the expression of turboRFP using a fluorescent microscope and the results were shown in FIG. 5B. In the absence of Doxycycline, none of the Zeocin selected cells produced turboRFP. Upon induction with Doxycycline, three out of ten isolated cell clones showed the phenotype of the turboRFP. These cell populations were identified as inducible clones. Their corresponding untreated cells were further cultured for use in other experiments.

### 5. Western Blot Analysis:

The corresponding untreated inducible cell aliquots were cultured in DMEM having 10% Tet-approved fetal bovine serum and subjected to analysis of GAPDH/V5 expression. 10⁶ of the cells were treated with 0.5 ug/ml Doxycycline for 48 hours and subjected to protein extraction using RIPA Lysis and Extraction Buffer (Thermo Scientific). Protein concentration in the lysate was determined using Protein Assay Dye Reagent Concentrate (Bio-Rad). 30 ug of protein samples were separated on a 12% SDS-PAGE gel and transferred to a nitrocellulose membrane (Bio-Rad). The membrane was incubated with 5% Blotting-Grade Blocker (Bio-Rad) for one hour at room temperature. GAPDH/V5 was immunodetected using HRP-mouse monoclonal anti-V5 antibody conjugate (Life Technologies) followed by SuperSignal West Pico Chemiluminescent Substrate (Thermo Scientific). Heat-shock protein-90 (HSP90) was used as an internal control. The HSP90 protein was detected by the mouse anti-human HSP90 alpha/beta monoclonal antibody (Sigma), followed by HRP-goat anti-mouse IgG conjugate (Santa Cruz) and SuperSignal West Pico Chemiluminescent Substrate. The result was shown by FIG. 5C. In the absence of Doxycycline, all 3 inducible clones did not produce the GAPDH/V5 protein, while treatment with Doxycycline resulted in the expression of GAPDH/V5 in all 3 cell clones.

### Example 5

### Inducible Expression of Human Glyceraldehyde-3-Phosphate Dehydrogenase (GAPDH) Gene in CHO-K1 Cells Using Intronic EGFP as an Indicator

### 1. Vector Construction:

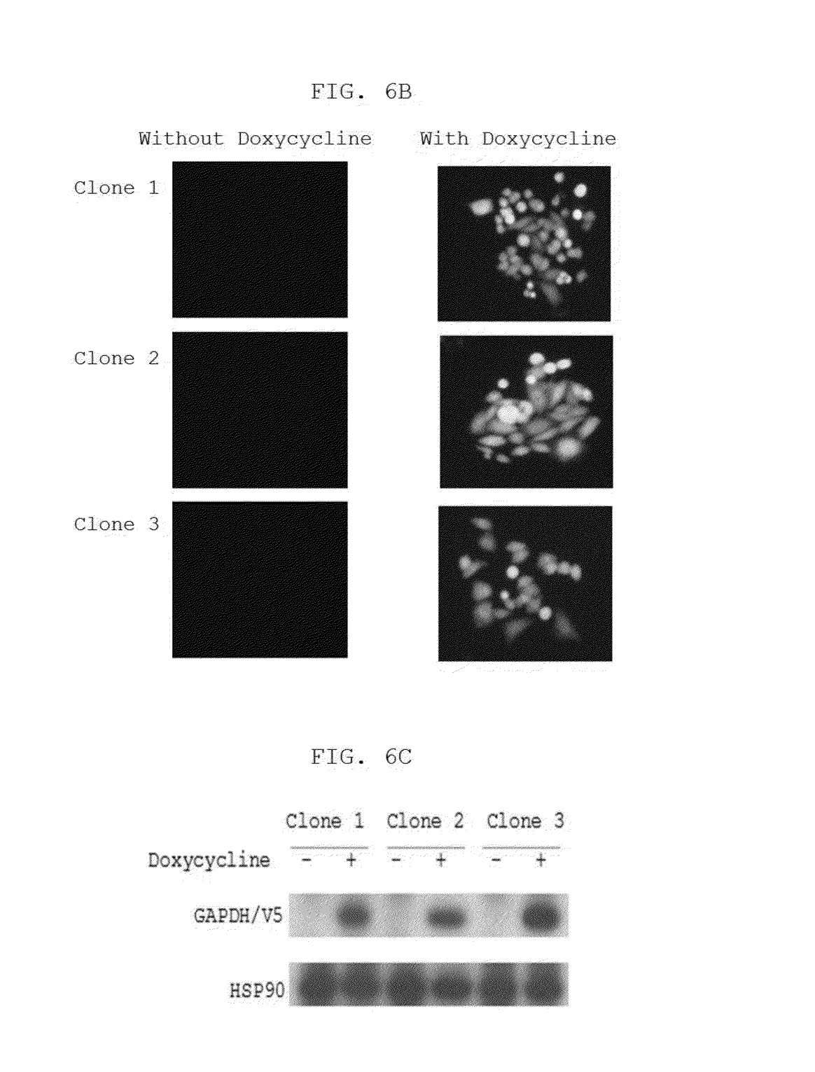

The use of the invention was further proven in other host cells. The lentiviral vector for inducible expression of GALDPH/V5 is illustrated by FIG. 6A. Expression of intronic EGFP and GAPDH/V5 was regulated by a linkage consisting of the tetracycline response element (TRE) and the minimal CMV promoter.

The intronic EGFP sequence was amplified by PCR using the pGR plasmid as a template. PCR primers also contained restriction sites used for further insertion into the pIGR-GAPDH/V5 lentiviral vector (i.e. AgeI HF site in 5′ position and EcoRI HF site in 3′ position).

Forward primer sequence:
(SEQ ID NO: 16)
5′-CTAATAACCGGTCTGGAGACGCCATCCACG-3′
Reverse primer sequence
(SEQ ID NO: 20)
5′-CATGATGAATTCGACGGTGACTGCAG-3′

The PCR product was digested by AgeI HF/EcoRI HF and inserted into the pIGR-GAPDH/V5 vector excised by the same restriction sites. The resulting pIGE-GAPDH/V5 plasmid was verified by DNA sequence analysis.

### 2. Preparation of Lentiviral Particles:

5×10⁵ of HEK293 cells in 2 ml of DMEM having 10% Tet-approved fetal bovine serum were added onto a 3.5-cm cell culture dish and cultured for 24 hours. Cells were co-transfected with 1.25 ug pIGE-GAPDH/V5, 0.94 ug psPAX, and 0.31 ug pMD2.G using the Lipofectamine LTX transfection reagent. Sixteen hours after transfection, cells were suspended in 5 ml DMEM having 10% Tet-approved fetal bovine serum and transferred to a 6-cm culture dish. After 48 hours, the culture medium was harvested and filtered through a 0.45 u filter. The filtered Lentiviral particles were stored at −80° C.

### 3. Stable Transfection of Chinese Hamster Ovary (CHO-K1) Cells:

Twenty-four hours prior to infection, 5×10⁵ CHO-K1 cells in DMEM/F12 medium having 10% Tet-approved fetal bovine serum were plated onto a 3.5-cm cell culture dish. After 24 hours, 50 uL of lentiviral particles and 8 ug/ml polybrene were added to the cells and incubated for 16 hours. The culture medium was removed and cells were suspended in 10 ml of fresh DMEM/F12 having 10% Tet-approved fetal bovine serum. Cells were transferred to a 10-cm cell culture dish. After 24 hours, 0.2 mg/ml Zeocin was added. Culture medium was replaced every 3 days. After 3 weeks, ten viable cell clones were isolated and split to 2 equal aliquots in 96-well cell culture plates. The cells were grown for 24 hours to allow attachment to the plate.

### 4. Microscope Analysis:

0.5 ug/ml of Doxycycline was added to one aliquot of cell clones to induce transgene expression. After culturing for 48 hours, cells were analyzed for the expression of EGFP using a fluorescent microscope and results were shown by FIG. 6B. In the absence of Doxycycline, cells did not produce EGFP. Upon induction by Doxycycline, three out of ten isolated single cell clones were capable of producing EGFP (green color). These cell populations were identified as inducible clones. Their corresponding untreated cells were cultured for use in other experiments.

### 5. Western Blot Analysis:

The inducible cell clones were cultured in DMEM/F12 medium having 10% Tet-approved fetal bovine serum and subjected to analysis of GAPDH/V5 expression. 10⁶ cells were treated with 0.5 ug/ml Doxycycline for 48 hours. Total proteins were extracted using RIPA Lysis and Extraction Buffer. Protein concentration in the lysate was determined using Protein Assay Dye Reagent Concentrate. 30 ug of protein samples were separated on a 12% SDS-PAGE gel and transferred to a nitrocellulose membrane. The membrane was incubated with 5% Blotting-Grade Blocker for 1 hour at room temperature. The GAPDH/V5 protein was immunodetected using HRP-mouse monoclonal anti-V5 antibody conjugate and SuperSignal West Pico Chemiluminescent Substrate. The heat-shock protein-90 (HSP90) was used as an internal control. The HSP90 protein was detected by the mouse anti-HSP90 monoclonal antibody (Abcam), followed by the HRP-goat anti-mouse IgG conjugate and SuperSignal West Pico Chemiluminescent Substrate.

The results were shown by FIG. 6B. In the absence of Doxycycline, none of these clones expressed GAPDH/V5, while incubation with Doxycycline triggered expression of GAPDH/V5 in all 3 inducible cell clones.

Taken together, the DNA construct and method provided by the invention is capable of producing desired products in a trackable manner. Because the fluorescent reporter is easily identified by a fluorescent microscope or a flow cytometer, only limited number of cells are needed for isolating and identifying a desired cell population. Therefore, the invention is capable of eliminating the need for amplification of transfected cell population to a large number prior to determining a positive one. In addition, the invention is suitable for both constitutive and inducible gene expression. The method provided by the invention is rapid, flexible, reliable, and convenient to perform.

While the foregoing written specification has been described in considerable detail with reference to certain preferred versions thereof, other versions are possible. Those skilled in the art will be able to carry out various changes, substitutions of equivalents, and alterations to the subject matter, without departing from the spirit and scope of the invention. It is therefore intended that any protection granted be limited only by the appended claims and their equivalents.

What is claimed is:

1. A recombinant DNA construct comprising in order from 5′ to 3′:
(a) a promoter;
(b) a fluorescent reporter gene positioned within an intron defined by a 5′-donor splice site comprising a splice donor sequence and a 3′-acceptor splice site comprising a splice acceptor sequence;
(c) a desired gene;
(d) a transcription terminator;
wherein the promoter regulates transcription of both the fluorescent reporter gene and the desired gene; wherein the transcription terminator is unique.

2. A polynucleotide comprising the DNA construct of claim 1.

3. A viral vector comprising the DNA construct of claim 2.

4. A recombinant virus comprising within its genome the DNA construct of claim 1.

5. A eukaryotic host cell comprising the DNA construct of claim 1.

6. A polynucleotide comprising the DNA construct of claim 1 for use in expressing the desired gene in a constitutive manner.

7. A polynucleotide comprising the DNA construct of claim 1 for use in expressing the desired gene in an inducible manner.

8. A method of producing a host cell comprising introducing a DNA construct of claim 1 into a population of host cells.

9. A host cell produced according to the method of claim 8.

10. A method of producing a desired protein comprising culturing a host cell of claim 9.

11. A method of producing a desired protein comprising isolating the protein from the culture of claim 10.
